(19) United States
(12) Patent Application Publication
(10) Pub. No.: US 2005/0277975 A1
(43) Pub. Date: Dec. 15, 2005

(54) Methods and apparatus for creating a working space within a body lumen or cavity

(75) Inventors: Vahid Saadat, Saratoga, CA (US); Chris Rothe, San Jose, CA (US); Ruey-Feng Peh, Sunnyvale, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: USGI Medical Corp., San Clemente, CA (US)

(21) Appl. No.: 10/865,686

(22) Filed: Jun. 9, 2004

(51) Int. Cl. A61M 29/00

(52) U.S. Cl. 606/191

(57) ABSTRACT

Methods and apparatus for creating a working space within a patient's stomach are described. The working space is created by internally spreading or pushing the walls of the stomach apart. Advantageously, such spreading is achieved without pressurizing the patient's stomach, i.e. without injecting a pressurized gas or other fluid directly into the stomach. Diagnostic or therapeutic instruments may be deployed within the working space. It is expected that spreading the walls without pressurizing the stomach will facilitate engagement and/or manipulation of the stomach wall for, e.g., endoluminal treatment of obesity.

### BACKGROUND OF THE INVENTION

Field of the Invention

The present invention relates to methods and apparatus for creating a working space or scaffolding within a body lumen or cavity. More particularly, the present invention describes methods and apparatus for creating a working space within, e.g., a patient's stomach, to facilitate endoluminal treatment of disorders.

Extreme or morbid obesity is a serious medical condition pervasive in the United States and other countries. Its complications include hypertension, diabetes, coronary artery disease, stroke, congestive heart failure, multiple orthopedic problems and pulmonary insufficiency with markedly decreased life expectancy.

Several surgical techniques have been developed to treat morbid obesity, including bypassing an absorptive surface of the small intestine, bypassing a portion of the stomach, and reducing or partitioning the stomach size, e.g., via Vertical Banded Gastroplasty (“VBG”) or Magenstrasse and Mill. These procedures may be difficult to perform in morbidly obese patients and/or may present numerous potentially life-threatening post-operative complications. Thus, less invasive techniques have been pursued.

U.S. Pat. Nos. 4,416,267 and 4,485,805 to Garren et al. and Foster, Jr., respectively, propose disposal of an inflated bag within a patient's stomach to decrease the effective volume of the stomach that is available to store food. Accordingly, the patient is satiated without having to consume a large amount of food. A common problem with these inflated bags is that, since the bags float freely within the patient's stomach, the bags may migrate to, and block, a patient's pyloric opening, the portal leading from the stomach to the duodenum, thereby restricting passage of food to the remainder of the gastro-intestinal tract.

Apparatus and methods also are known in which an adjustable elongated gastric band is laparoscopically disposed around the outside of a patient's stomach near the esophagus to form a collar that, when tightened, squeezes the stomach into an hourglass shape, thereby providing a stoma that limits the amount of food that a patient may consume comfortably. An example of an adjustable gastric band is the LAP-BAND® made by INAMED Health of Santa Barbara, Calif.

Numerous disadvantages are associated with using an adjustable gastric band. First, the band may be dislodged if the patient grossly overeats, thereby requiring additional invasive surgery to either reposition or remove the band. Similarly, overeating may cause the band to injure the stomach wall if the stomach over-expands. Laparoscopic disposal of the gastric band around the stomach requires a complex procedure, requires considerable skill on the part of the clinician, and is not free of dangerous complications.

Endoluminal treatment of obesity, whether performed transorally or transgastrically (or a combination thereof), is expected to require engagement and manipulation of tissue within the gastric lumen. The gastric lumen may be insufflated to create a working space within the stomach to facilitate such engagement and manipulation. However, insufflation requires that a good seal be formed in the stomach to ensure sufficient expansion of the stomach. Furthermore, insufflation imparts stress on the stomach wall, which is akin to the hoop stress observed in a pressurized cylinder. This stress compresses tissue layers making up the gastric wall and provides a restoring force that complicates attempts to grasp or engage the stomach wall from its interior.

In view of the drawbacks associated with conventional techniques for creating a working space within the stomach, it would be desirable to provide methods and apparatus that overcome those drawbacks.

### BRIEF SUMMARY OF THE INVENTION

A working space is created within a patient's stomach by internally spreading or pushing the walls of the stomach apart. Advantageously, such spreading is achieved without pressurizing the patient's stomach, i.e. without injecting a pressurized gas or other fluid directly into the stomach. Diagnostic or therapeutic instruments may be deployed within the working space formed within the stomach. It is expected that spreading the walls of the stomach without pressurizing the stomach will facilitate engagement and/or manipulation of the stomach wall for, e.g., endoluminal treatment of obesity.

In one variation, an expandable mechanical device is provided to spread the stomach walls and create a working space. The mechanical device provides a scaffold, platform, structure or other support that maintains the working space. The device may be advanced into the gastric lumen transorally or, alternatively, may be advanced into the lumen transgastrically. In one variation, diagnostic or therapeutic instruments optionally may be coupled to the scaffold, for example, to enhance functionality, allow for deployment of additional instruments, to aid treatment, etc.

### BRIEF DESCRIPTION OF THE DRAWINGS

FIGS. 1A-1C are schematic views of apparatus for creating a working space within a patient's stomach and methods for its deployment.

FIGS. 2A-2D are side views, partially in section, illustrating a method of using the apparatus of FIG. 1 to create a working space within a patient's stomach via a transoral approach.

FIGS. 3A-3C are side views, partially in section, illustrating a method of using the apparatus of FIG. 1 to create a working space in an alternative plane of the patient's stomach.

FIG. 4 is a schematic perspective view illustrating an alternative method of deploying the apparatus of FIG. 1.

FIGS. 5A and 5B are side views illustrating another alternative method for deploying the apparatus of FIG. 1.

FIG. 6 is a schematic perspective view of a variation of the apparatus.

FIGS. 7A and 7B are side-sectional views illustrating a method of using the apparatus of FIG. 6 to create a working space.

FIG. 8 is a schematic perspective view of another variation of the apparatus.

FIGS. 9A and 9B are side-sectional views illustrating a method of using the apparatus of FIG. 8 to create a working space.

FIG. 10 is a schematic perspective view of yet another variation of the apparatus.

FIGS. 11A and 11B are side-sectional views illustrating a method of using the apparatus of FIG. 10 to create a working space.

FIGS. 12A and 12B are side views of yet another variation of the apparatus in a collapsed delivery configuration, and in an expanded deployed configuration.

FIGS. 13A and 13B are side-sectional views illustrating a method of using the apparatus of FIG. 12 to create a working space.

FIG. 14 is a side-sectional view illustrating a method of using a variation of the apparatus of FIG. 12 to create a working space and to deploy instruments into the working space.

FIG. 15 is a detail view of a tissue-engaging element for use with apparatus of the present invention.

FIGS. 16A-16C are side views, partially in section, illustrating a method of creating a working space within a patient's stomach utilizing the apparatus of FIG. 15 in conjunction with the apparatus of FIG. 1.

FIGS. 17A and 17B are detail views of an alternative tissue-engaging element in an open and a closed configuration.

FIGS. 18A and 18B are side-sectional views illustrating a method of creating a working space with the apparatus of FIGS. 17 in conjunction with the apparatus of FIG. 1.

FIG. 19 is a detail view of another alternative tissue-engaging element;

FIGS. 20A and 20B are side-sectional views illustrating a method of creating a working space with the apparatus of FIG. 19 in conjunction with the apparatus of FIG. 1.

FIG. 21 is a detail view of yet another alternative tissue engaging element;

FIGS. 22A and 22B are side-sectional views illustrating a method of creating a working space with the apparatus of FIG. 21 in conjunction with the apparatus of FIG. 1.

FIG. 23 is a detail view of still another alternative tissue engaging element;

FIGS. 24A and 24B are side-sectional views illustrating a method of creating a working space with the apparatus of FIG. 23.

FIG. 25 is a schematic perspective view of apparatus for transgastrically creating a working space within a patient's stomach.

FIGS. 26A and 26B are side views illustrating a method of using the apparatus of FIG. 25 to transgastrically create a working space within a patient's stomach.

FIG. 27 is a side view, partially in section, illustrating a method of using the apparatus of FIG. 25 in combination with the apparatus of FIG. 1 to create a working space within a patient's stomach.

FIGS. 28A and 28B are schematic perspective views of apparatus for creating a working space, shown, respectively, advanced through a trocar for transgastric actuation and advanced through an endoscope for transoral actuation.

FIGS. 29A and 29B are side views illustrating a method of using the apparatus of FIG. 28A to transgastrically create a working space within a patient's stomach.

### DETAILED DESCRIPTION OF THE INVENTION

Methods and apparatus for creating a working space within a patient's stomach are described. The working space is created by internally spreading or pushing the walls of the stomach apart. Advantageously, such spreading is achieved without pressurizing the patient's stomach, i.e. without injecting a pressurized gas or other fluid directly into the stomach. Diagnostic or therapeutic instruments may be deployed within the working space. It is expected that spreading the walls without pressurizing the stomach will facilitate engagement and/or manipulation of the stomach wall for, e.g., endoluminal treatment of obesity, as well as reduce the complications typically associated with conventional insufflation procedures.

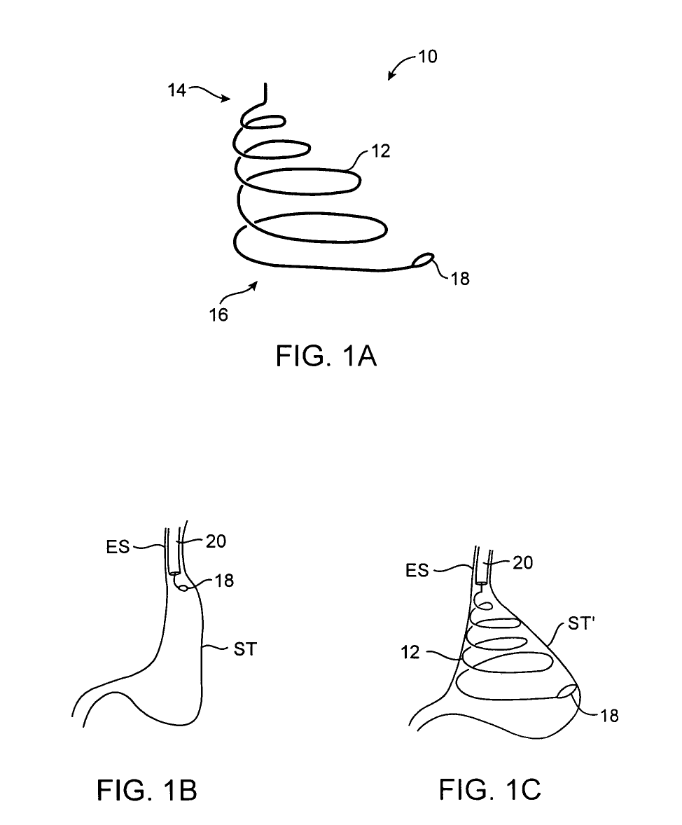

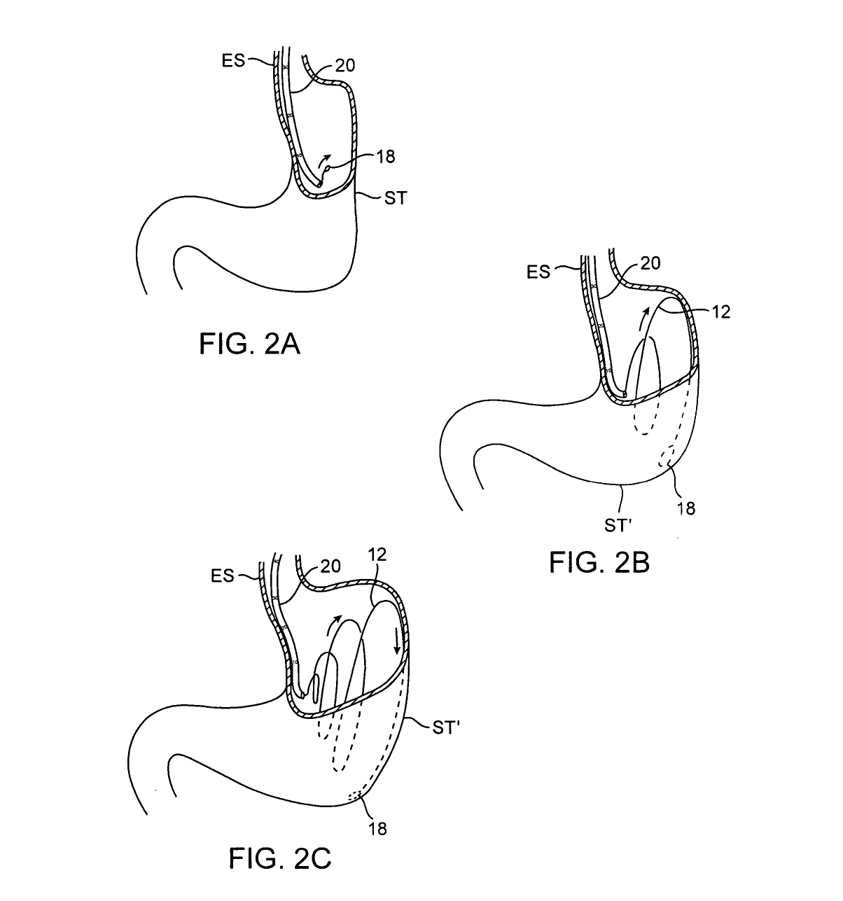

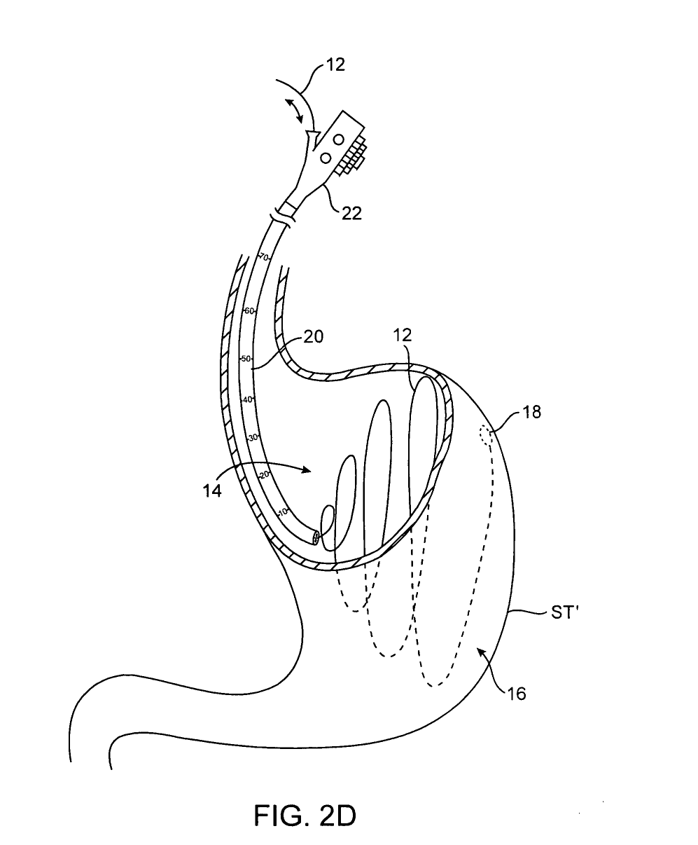

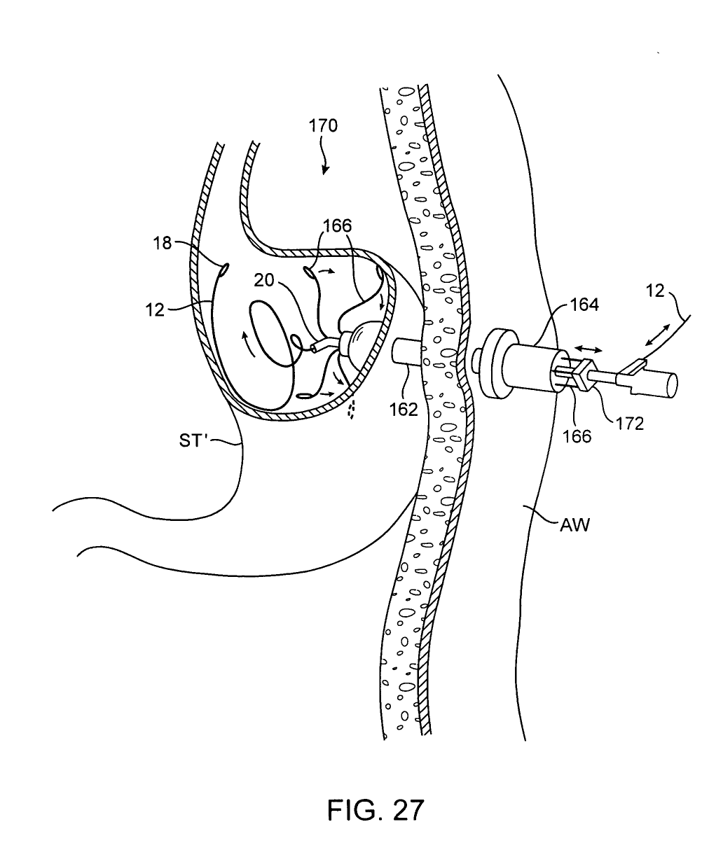

Referring now to FIG. 1, a variation of the apparatus is described. Apparatus 10 comprises shape memory wire or ribbon 12 having an expanded profile adapted to endoluminally spread the walls of a patient's stomach. Apparatus 10 may provide a scaffold, platform, structure or other support that maintains a working space within the patient's stomach. Apparatus 10 may be advanced into the gastric lumen transorally, as described hereinbelow with respect to FIGS. 2, or, alternatively, may be advanced into the lumen transgastrically, as described hereinbelow with respect to FIG. 27. Diagnostic or therapeutic instruments optionally may be coupled to the scaffold provided by apparatus 10 within a patient's stomach, for example, to enhance functionality, allow for deployment of additional instruments, to aid treatment, etc.

Wire 12 of apparatus 10 illustratively comprises a spiral or helix shape that may be configured to increase in diameter from the proximal region 14 towards the distal region 16 of the expandable portion of wire 12, such as in a conically-shaped helix. Apparatus 10 preferably is fabricated from a shape memory material, such as Nitinol, such that the apparatus may be advanced through, e.g., a tube, sheath, catheter, or endoscope lumen in a collapsed delivery configuration, and then may dynamically expand to expanded deployed configuration of FIG. 1 after exiting a distal end of the constraining tube, sheath, etc. A distal end of wire 12 may also comprise an atraumatic end 18, e.g., a blunted distal end, looped portion, capped with a soft material, etc., to prevent tearing or ripping of the tissue.

FIGS. 1B and 1C show partial cross-sectional views of the apparatus 10 of FIG. 1A in one variation of how the device may be utilized. The distal end of a delivery device 20, e.g., endoscope, catheter, sheath, etc., may be advanced transorally past the esophagus ES and into a stomach ST to be expanded, as shown in FIG. 1B. Alternatively, the distal end may be advanced transorally just proximally of or at esophagus ES. While delivery device 20 is held stationary relative to stomach ST, wire 12 may then be advanced distally to exit from delivery device 20. In the case of an endoscope being utilized, wire 12 may simply be delivered through one of the working channels of the endoscope leaving the other working channels open for the delivery of other tools. As wire 12 exits from device 20, it may begin to self-expand once free from the constraints of device 20 and reconfigure itself into its expanded profile. As wire 12 continues to expand and advance distally, the stomach ST may be expanded via wire 12 until the desired region of tissue within expanded stomach ST′ has been cleared to provide a sufficient working area. Atraumatic wire end 18 may prevent wire 12 from tearing or puncturing the tissue as wire 12 is advanced into the stomach. Once a procedure has been completed within the cleared working area, wire 12 may be simply withdrawn proximally into delivery device 20 and removed entirely from the patient.

The example of FIGS. 1B and 1C demonstrate how the apparatus 10 may be deployed from the delivery device 20 positioned distally of esophagus ES. In such an orientation, wire 12 may be expanded to provide a working area near or at the antrum of the expanded stomach ST′. By simply repositioning delivery device 20, other regions of the stomach may be selectively expanded. For instance, FIGS. 2A to 2C illustrate how delivery device 20 may be advanced further into unexpanded stomach ST.

Once positioned within stomach ST, as in FIG. 2A, wire 12 may be deployed to begin clearing the region of stomach along the greater curvature, as seen in FIG. 2B. FIG. 2C shows the wire 12 having been fully deployed and the stomach ST′ expanded as desired. FIG. 2D shows a partial cross-sectional view of delivery device 20 and expanded wire 12 fully deployed within expanded stomach ST′. Wire 12 may be manipulated and advanced (or withdrawn) from delivery device 20 via its proximal end through handle 22 of delivery device 20.

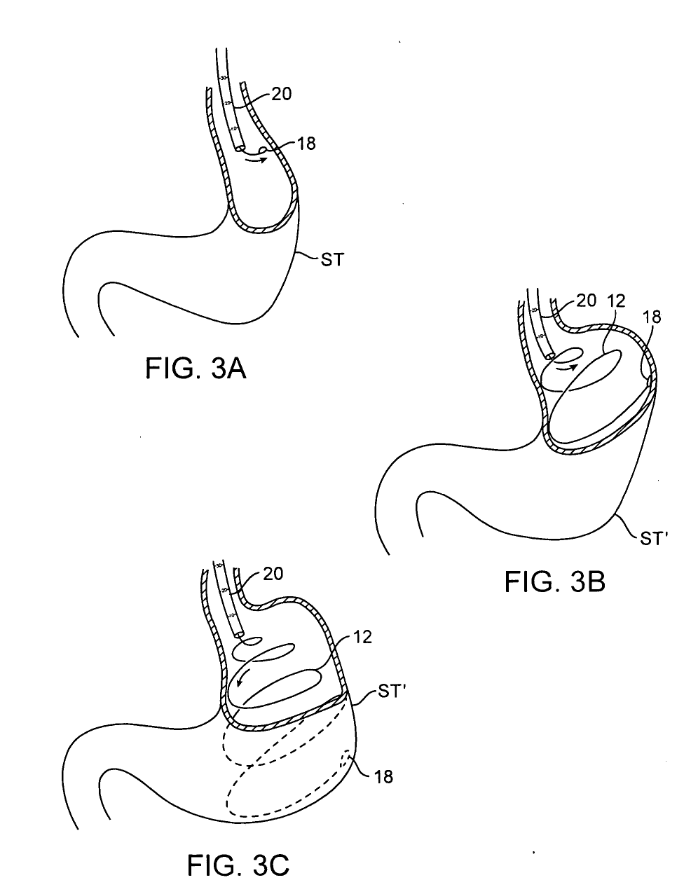

FIGS. 3A to 3C show partial cross-sectional views of yet another method in which apparatus 10 may be used to clear a portion of an anterior region of the stomach. As above, delivery device 20 may be advanced into stomach ST, as shown in FIG. 3A, and wire 12 may be deployed, as shown in FIG. 3B, such that an anterior portion of the stomach ST′ is cleared and expanded, as shown in FIG. 3C.

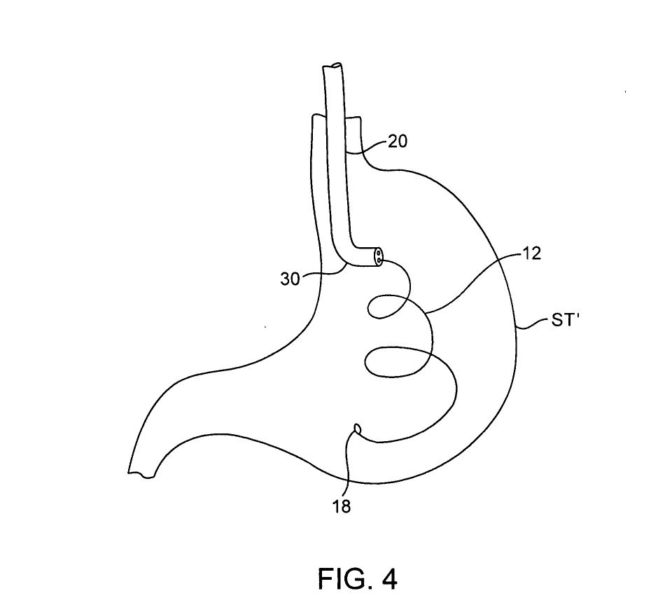

Because the wire 12 exiting the delivery device 20 may be inclined to exit into the stomach at an undesirable angle before the wire 12 is able to reconfigure itself, delivery device 20 may include an articulatable portion 30, as typically seen in conventional endoscopes as in FIG. 4. Articulatable portion 30 may be angled such that the wire 12 is able to exit the device 20 substantially straight and then reconfigure itself as it expands distally relative to the delivery device 20 into the stomach ST′.

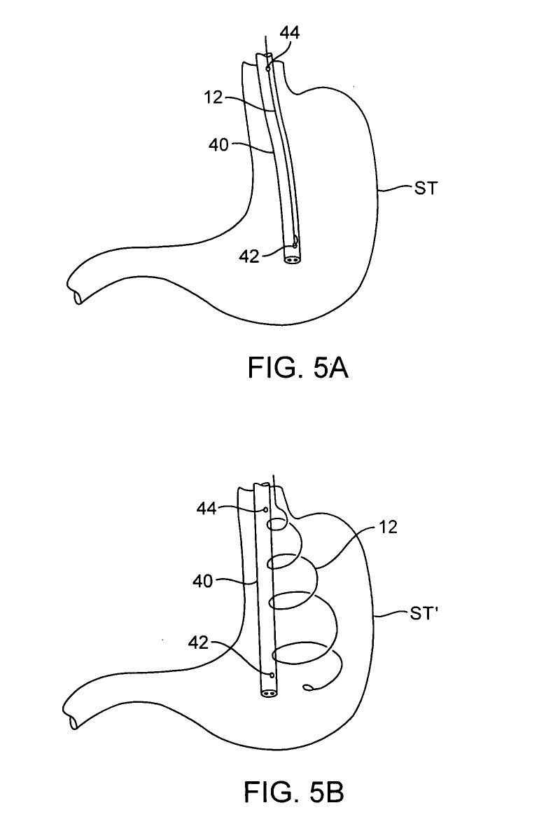

Rather than being delivered through the delivery device, wire 12 may be delivered while adjacently attached to the device. As shown in FIG. 5A, a modified delivery device 40 may carry wire 12 attached onto the outer surface of device 40 into stomach ST. Wire 12 may be temporarily connected to device 40 at two or more attachment points 42, 44. Once the device 40 has been advanced sufficiently into the lumen of stomach ST, attachment points 42, 44 may be actuated to release wire 12 from device 40. Once wire 12 has been released, it may then be allowed to reconfigure itself simultaneously such that the stomach ST′ expands as well and device 40 may be removed. To withdraw wire 12 from the stomach, delivery device 40 may be advanced back into the stomach ST′ while withdrawing wire 12 through one of its working channels or a sheath may simply be advanced into stomach ST′ while withdrawing wire 12 therethrough.

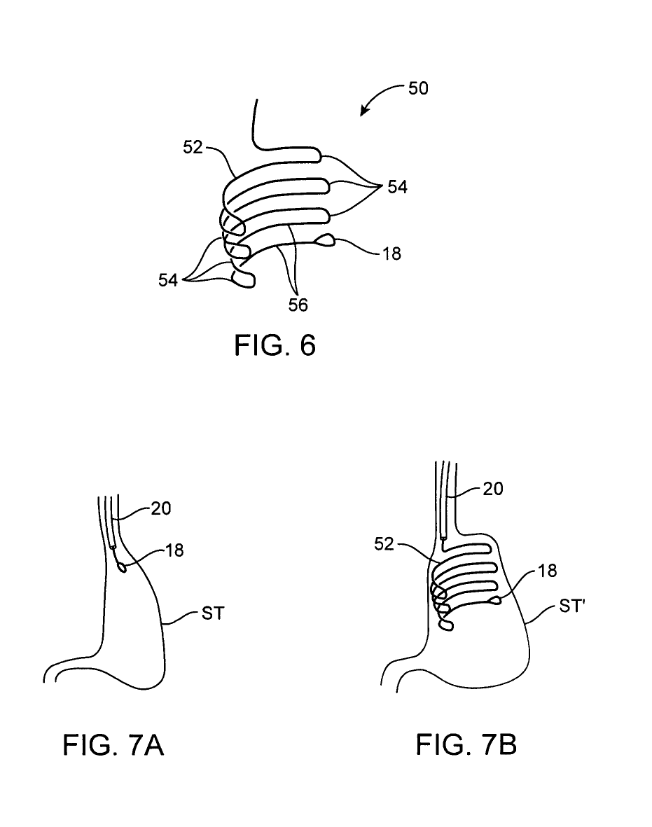

FIG. 6 shows a perspective view of an expandable apparatus 50 having an alternative expanded configuration. Expanded apparatus 50 may generally comprise ribbon or wire 52 configured to define multiple curved sections 56 of wire patterned in an alternating manner around bend portions 54. As shown in FIG. 7A, apparatus 50 may be deployed into stomach ST; as wire 52 is advanced and assumes its expanded configuration, curved sections 56 of wire 52 may be positioned against lesser curvature of stomach ST′, or any other portion of the stomach ST′, such that stomach ST′ assumes a tented shape.

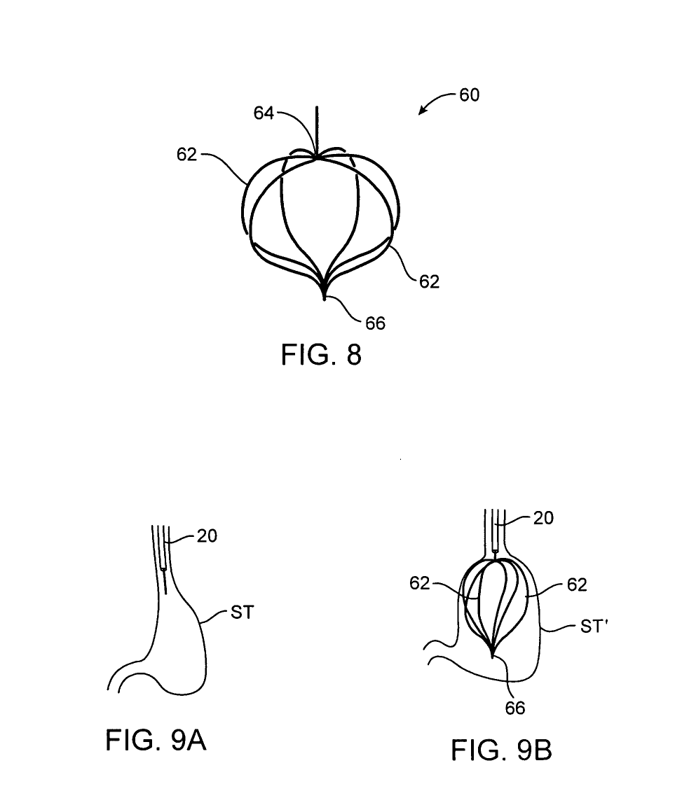

FIG. 8 shows a perspective view of expandable apparatus 60 having yet another alternative configuration. Apparatus 60 may assume, in this example, a spherical or ellipsoid shape with multiple expandable arms or struts 62 joined proximally and distally at respective attachment points 64, 66. In alternative configurations, the apparatus may be configured to assume other shapes, e.g., cylindrical, conical, etc. In use, apparatus 60 may be advanced through delivery device 20 in stomach ST, as shown in FIG. 9A, and then fully deployed within the stomach such that apparatus 60 expands stomach ST′ into an open configuration to expose the region of tissue of interest, as in FIG. 9B.

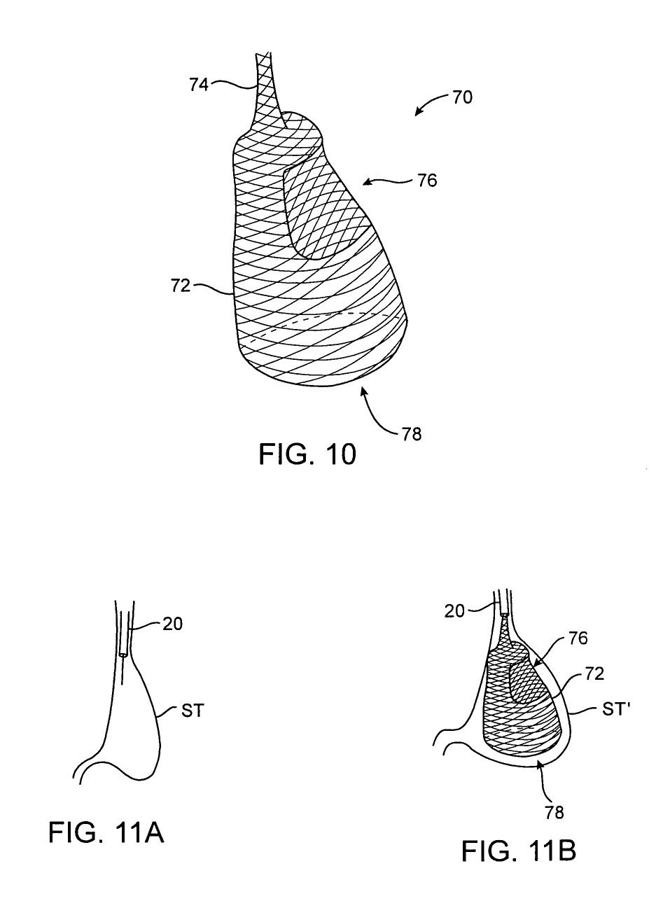

FIG. 10 shows a perspective view of yet another variation in apparatus 70. In this variation, apparatus 70 may generally comprise an expandable mesh or netting 72 extending from an elongate section 74. Mesh 70 may be fabricated from a plurality of interwoven shape memory alloy wires, such as Nitinol as described above. Moreover, mesh 70 may be configured to expand into a conical shape such that an opening 78 may be optionally defined at a distal region of mesh 70. An additional opening 76 may also be defined along the surface of mesh 70.

Mesh 70 may be advanced into stomach ST, as described above and as shown in FIG. 11A. Once deployed, mesh 70 may be expanded to tent the stomach ST′ into an expanded configuration. Tools may then be deployed through a lumen defined in elongate section 74 and advanced towards opening 78 or opening 76 to work on the tissue defined by either of those openings. Alternatively, tools may be advanced transgastrically into the stomach through a gastrostomy and through opening 76 from outside the patient to work on the tissue defined by opening 78, if desired. To remove mesh 70 from the patient, it may simply be withdrawn into delivery device 20 and removed from the stomach.

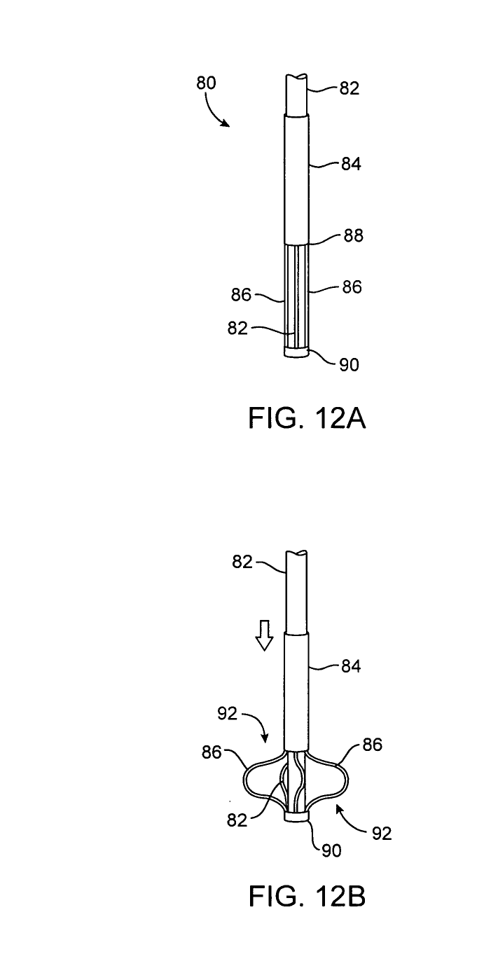

FIGS. 12A and 12B show perspective views of yet another expandable apparatus 80. FIG. 12A shows a perspective view of apparatus 80 generally comprising inner tube 82 and outer tube 84, which may be slidably disposed over inner tube 82. Outer tube 84 may have a plurality of reconfigurable arms or struts 86 connected to the distal end of outer tube 84 at attachment points 88 and to the distal end of inner tube 82 at attachment points 90. Arms 86 may generally comprise a ribbon or wire spaced adjacent to one another preferably in a parallel configuration, although this is not necessary. To expand reconfigurable arms 86 into an enlarged configuration, outer tube 84 may be advanced distally in the direction of the arrow shown in FIG. 12B relative to inner tube 82. Alternatively, inner tube 82 may be withdrawn proximally or both inner tube 82 and outer tube 84 may be advanced relative to one another. Arms 86 may be made from a variety of materials such as shape memory alloys, stainless steels, plastics, etc.

Although this variation may be manipulated by the user to expand reconfigurable arms or struts 86, it may alternatively be configured to have arms or struts 86 to be self-expanding. In such a case, arms or struts 86 may be made from a shape memory alloy such as Nitinol, as described above. Moreover, outer tube 84 may be used to constrain arms 86 in a straightened configuration until the apparatus 80 has been desirably positioned within the stomach. Once arms 86 are ready for release, outer tube 84 may be simply released from a locked configuration to allow arms 86 to self-expand. As the arms 86 expand, openings or spaces 92 are formed between the arms 86.

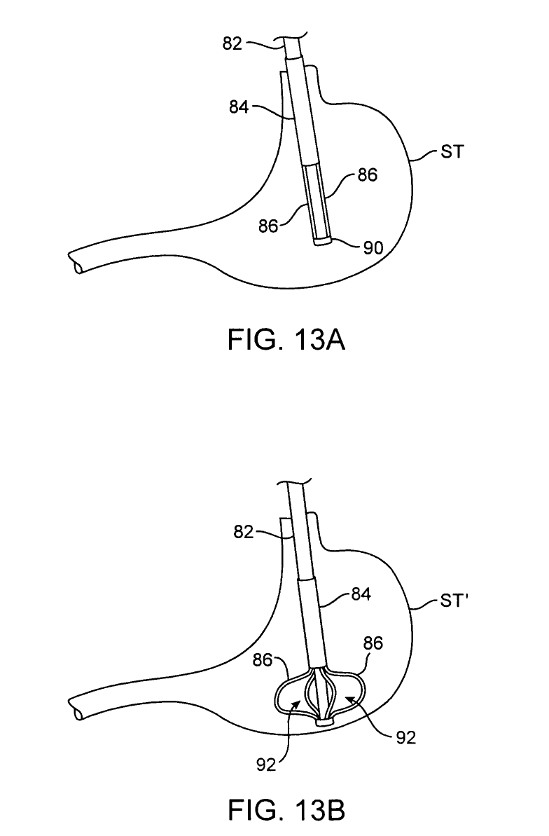

FIGS. 13A and 13B show partial cross-sectional views of the apparatus of FIGS. 12A and 12B within stomach ST. Once the apparatus 80 has been advanced into an unexpanded stomach ST, as shown in FIG. 13A, reconfigurable arms 86 may be actively expanded or allowed to self-expand, depending upon which variation is utilized, as shown in FIG. 13B. Once fully expanded, the openings or spaces 92 may provide working areas to allow for tool access to the tissue, as shown.

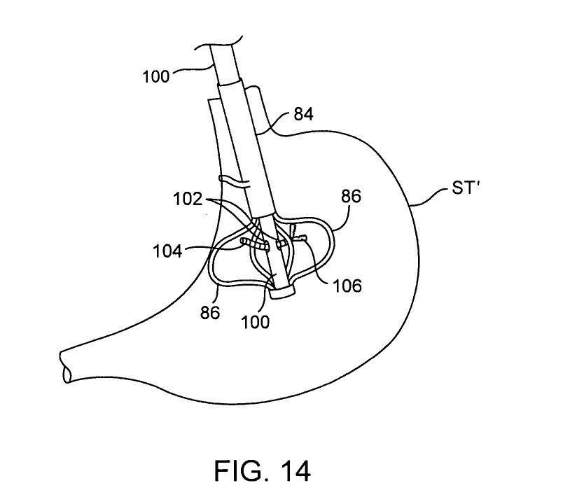

FIG. 14 shows a partial cross-sectional view of a variation of the apparatus of FIGS. 12A and 12B. As seen, outer tube 84 may be allowed to slide over a modified inner tube 100. Once reconfigurable arms 86 have been expanded, one or more openings 102 defined over the surface of inner tube 100 may be exposed. Openings 102 may be defined along the region of inner tube 100 which is adjacent to arms 86. Once arms 86 have expanded to displace the tissue from the region of interest, one or more tools 104, 106 may be advanced down inner tube 100 to emerge from openings 102 to work upon the tissue region of interest. The reconfigured arms 86 thus provide a defined area or opening for access to the tissue within which the tools 104, 106 may be utilized. Tools 104, 106 may generally comprise any tools as practicable, e.g., imagers, light fibers, graspers, cutting tools, etc.

As described above, the delivery device or endoscope 20 may be held stationary relative to the stomach and the expandable apparatus may be advanced distally relative to the delivery device 20. Alternatively, the delivery device 20 may be advanced into the stomach towards the tissue region of interest and the expandable apparatus may be advanced distally out of device 20 while the device 20 is then withdrawn proximally relative to the ribbon or wire. The distal end of the apparatus may be left float freely within the stomach during this procedure. Alternatively, the distal end may be temporarily anchored into the tissue region of interest so that the distal end remains stationary while the delivery device is withdrawn proximally.

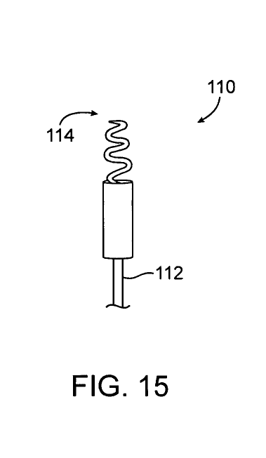

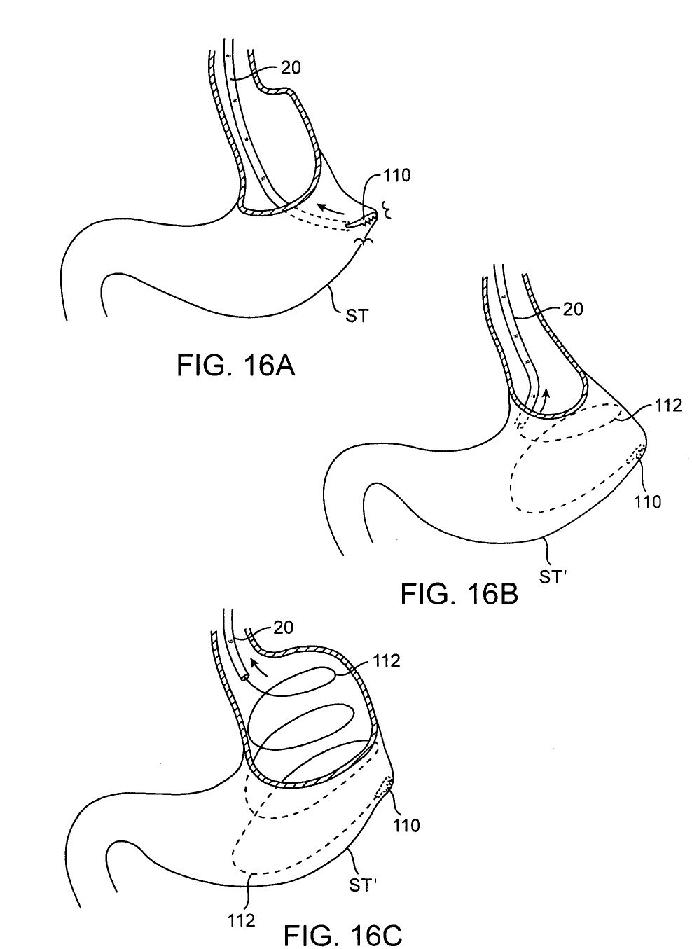

FIG. 15 shows a side view of one variation of an anchoring distal end 110 for the expandable apparatus. As shown, piercing helix 114 may be disposed at the end of ribbon or wire 112 for anchoring into the tissue. FIG. 16A shows a partial cross-sectional view of delivery device 20 having been advanced into stomach ST towards the tissue region of interest. Anchoring distal end 110 may be advanced out of delivery device 20 and torqued into the tissue. Once the distal end 110 has been sufficiently anchored to the tissue, delivery device 20 may be withdrawn proximally while allowing wire 112 to slide out of device 20, as shown in FIG. 16B. FIG. 16C shows wire 112 fully deployed and stomach ST′ in an expanded condition. To remove wire 112, delivery device 20 may be advanced distally back into stomach ST′ while drawing wire 112 within. Once delivery device 20 has been advanced to anchoring distal end 110, the helix 114 may be torqued out of the tissue and device 20 may be removed.

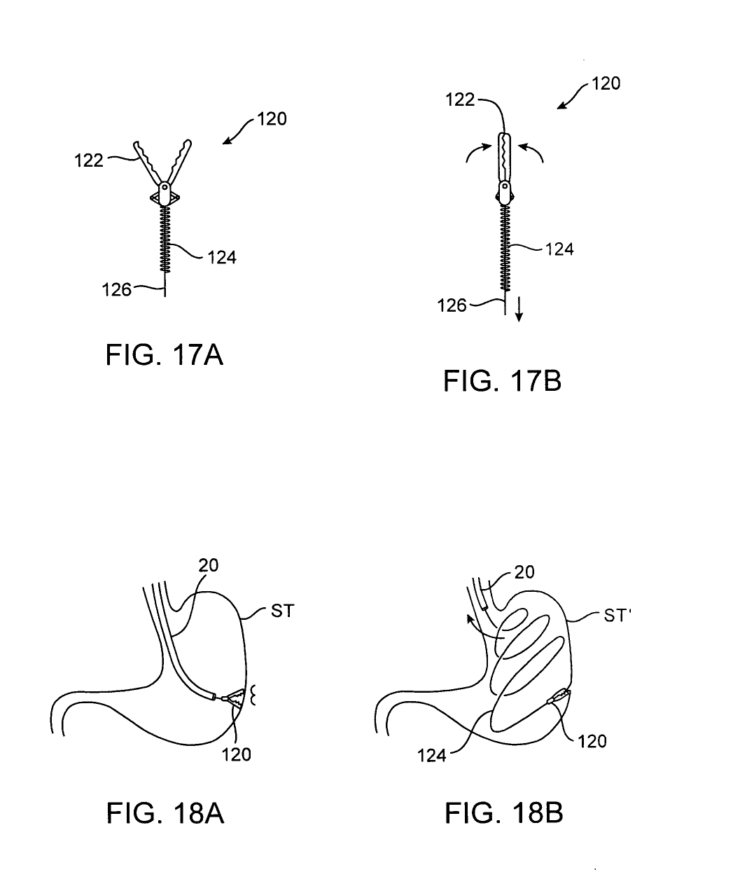

FIGS. 17A and 17B show side views of another variation for anchoring the ribbon or wire. Anchoring distal end 120 may be configured in this variation as a grasper 122, shown in its open configuration in FIG. 17A and closed configuration in FIG. 17B. Grasper 122 may be disposed on the distal end of a tubular member 124 and manipulated via a control wire 126 routed therethrough for manipulating the jaws of grasper 122. Tubular member 124 may also be fabricated from a shape memory material, such as Nitinol as described above, to enable the expansion of stomach tissue.

As shown in FIG. 18A, delivery device 20 may be advanced into the stomach ST towards the tissue region of interest, as above, and grasper 120 may be advanced out of device 20 and clamped onto the tissue region of interest. Once grasper 120 has been sufficiently anchored to the tissue, device 20 may be withdrawn proximally from stomach ST′ while allowing tubular member 124 to extend out of device 20 and reconfigure to expand the stomach ST′, as shown in FIG. 18B. Removal of the tubular member 124 may be accomplished in the same manner as described above. Alternatively, grasper 122 may be simply articulated to release the tissue; then tubular member 124, along with grasper 122, may be simply withdrawn proximally into the delivery device 20 for removal from the patient.

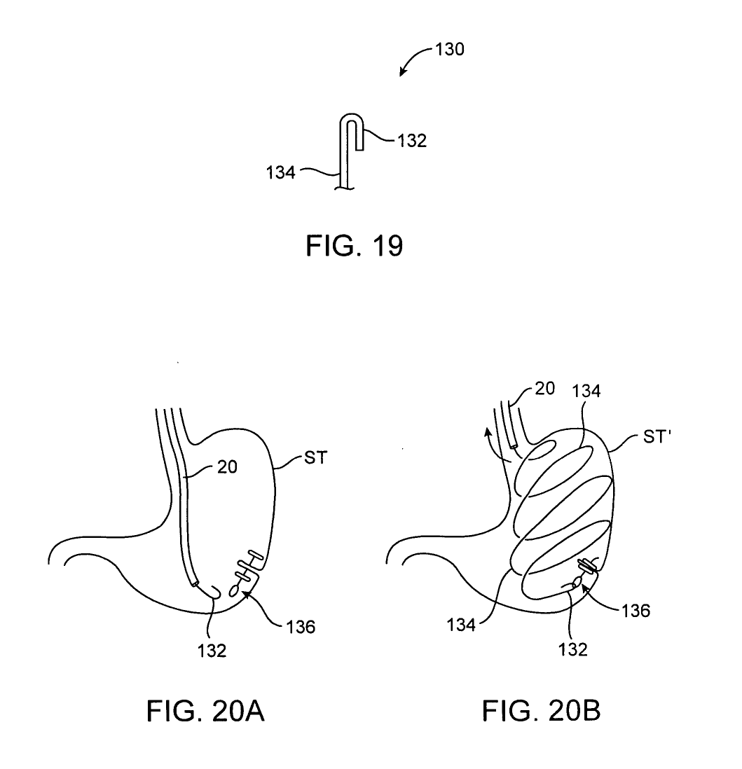

FIG. 19 shows a side view of yet another variation of anchoring distal end 130. This variation may generally comprise a distal end of ribbon or wire 134 configured into a hook member 132. As shown in FIG. 20A, hook member 132 may be temporarily hooked onto a suture 136 extending from a tissue plication assembly. The tissue plication may be pre-formed using various conventional techniques and tools. The tissue plication may also be formed utilizing the tools and methods as described in co-pending U.S. patent application Ser. No. 10/735,030 filed Dec. 12, 2003, which is incorporated herein by reference in its entirety.

Once hook member 132 has been anchored to the suture, delivery device 20 may be withdrawn while allowing wire 134 to be deployed to expand the stomach ST′, as shown in FIG. 20B. Removal of wire 134 may be accomplished in the manner as described above.

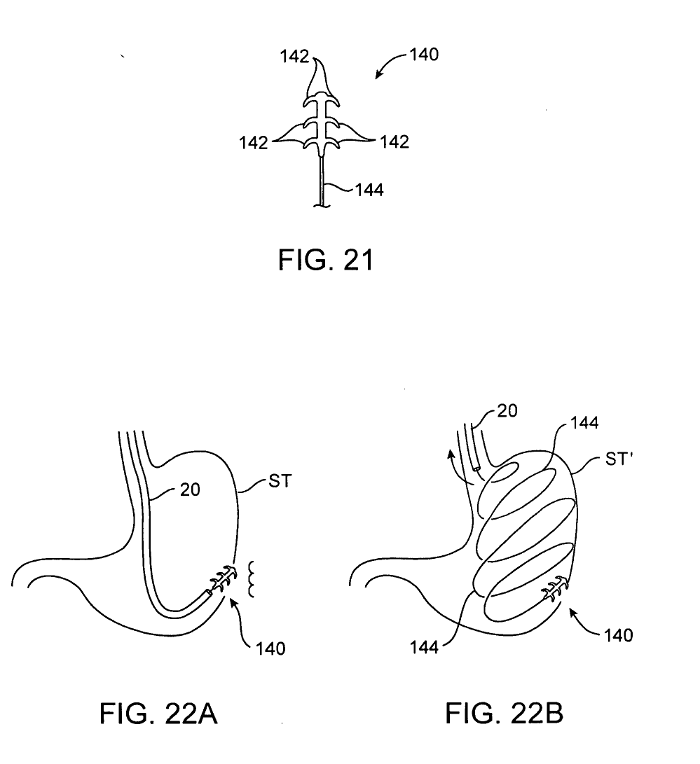

FIG. 21 shows a side view of yet another variation of anchoring distal end 140. This variation generally comprises a plurality of hooks or barbs 142 projecting from a distal end of ribbon or wire 144 for attachment to a tissue surface. As shown in FIG. 22A, anchoring distal end 140 may be advanced out of delivery device and against the tissue region of interest. Anchoring end 140 may be manipulated to ensure attachment onto the tissue, if desired. With anchoring end 140 attached to the tissue, delivery device 20 may be withdrawn proximally while allowing wire 144 to be deployed and expand within stomach ST′. Removal of wire 144 may be accomplished in the same manner as described above.

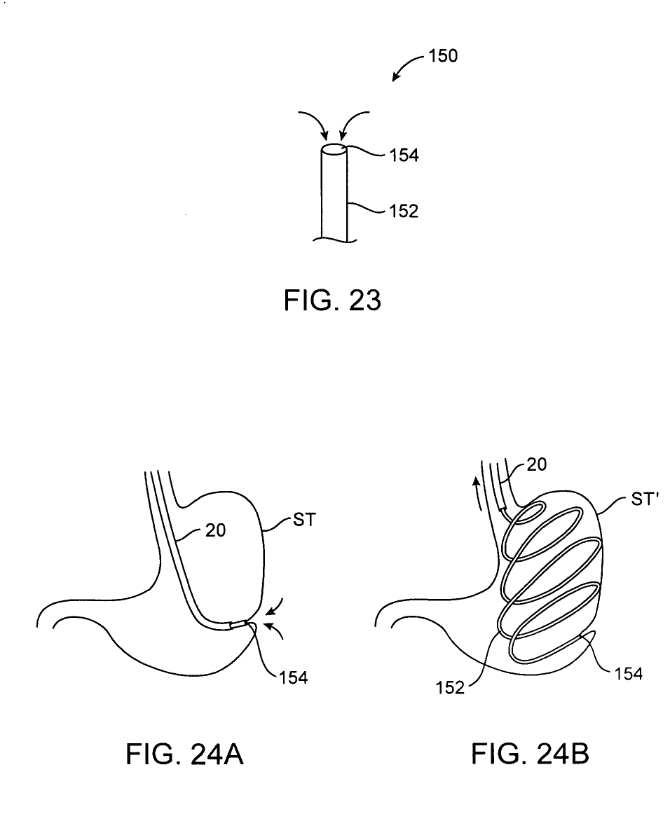

FIG. 23 shows an anchoring distal end 150 of another variation which may utilize a vacuum force. As illustrated, a vacuum force may be created within opening 154 at the distal end of reconfigurable tubular member 152. Rather than a single opening 154, a plurality of openings may be defined along the length of tubular member 152 near or at the distal end of member 152. Moreover, tubular member 152 may also be fabricated from a shape memory material, such as Nitinol as described above, to enable the expansion of stomach tissue. In use, delivery device 20 may be advanced into the stomach ST and anchoring distal end 150 may be advanced distally, as shown in FIG. 24A. The vacuum force may be activated within tubular member 152 such that opening 154 becomes attached to the tissue surface. While attached, tubular member 152 may be advanced out of delivery device 20 and allowed to expand within stomach ST′. To remove tubular member 152, the vacuum may be stopped and the tubular member 152 may be simply removed from the stomach through delivery device 20.

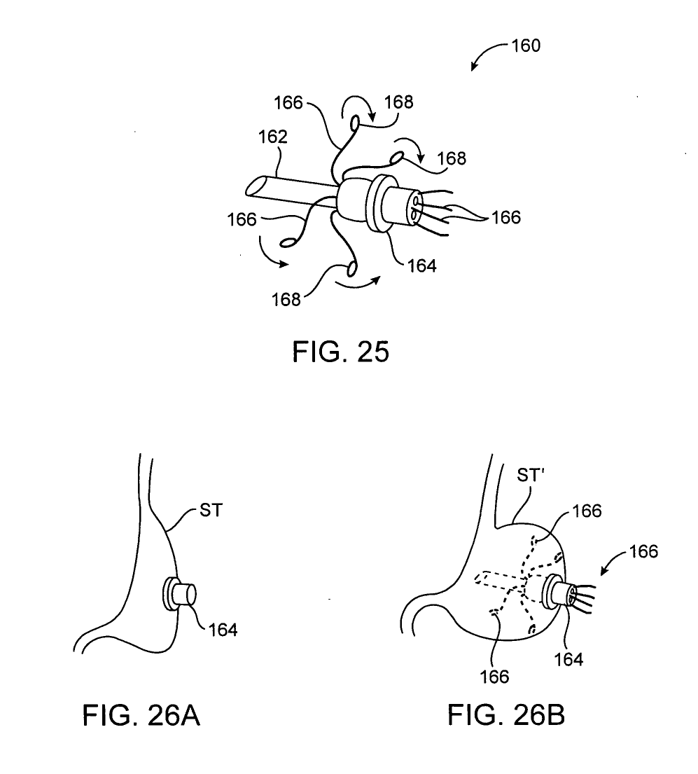

Aside from expansion devices being deliverable through some delivery device or endoscope lumen, other variations may be utilized for stomach expansion. For instance, FIG. 25 shows a perspective view of one variation which utilizes a transgastric approach into the stomach in expandable insert assembly 160. A trocar, conventional or specially designed, may be positioned within a laparoscopic port 164. A plurality of reconfigurable arms 166 may also extend through port 164 either independently of one another or through trocar 162. Arms 166, preferably made from a shape memory alloy such as Nitinol, may each comprise an atraumatic distal end 168 and is preferably configured to deploy itself at an angle relative to a longitudinal axis of the port 164, as shown by the arrows in FIG. 25. Each of the arms 166 may accordingly be positioned relative to one another to extend away from one another once positioned through port 164 such that the stomach tissue around port 164 becomes tented open and creates a working space free of tissue.

As shown in FIG. 26A, port 164 may be positioned through a gastrostomy to extend into stomach ST. The trocar 162 and/or arms 166 may then be advanced into stomach ST and allowed to reconfigure such that the stomach ST′ becomes tented and open, as shown in FIG. 26B.

FIG. 27 shows the device of FIG. 25 positioned through the skin and abdominal wall AW of a patient and extending through a gastrostomy into the stomach ST′ along with a delivery device 20 in a combination assembly 170. Reconfigurable arms 166 may be seen as being manipulatable from outside the patient via arm advancement assembly 172 which may be used to connect each of the arms 166 to one another to enable the uniform advancement or withdrawal of the arms 166. Delivery device 20 may also be seen to extend through port 164 and extend into stomach ST′ to deploy wire 12 into the stomach to further expand the stomach, if so desired. Removal of either or both devices may be accomplished in the manner as described above.

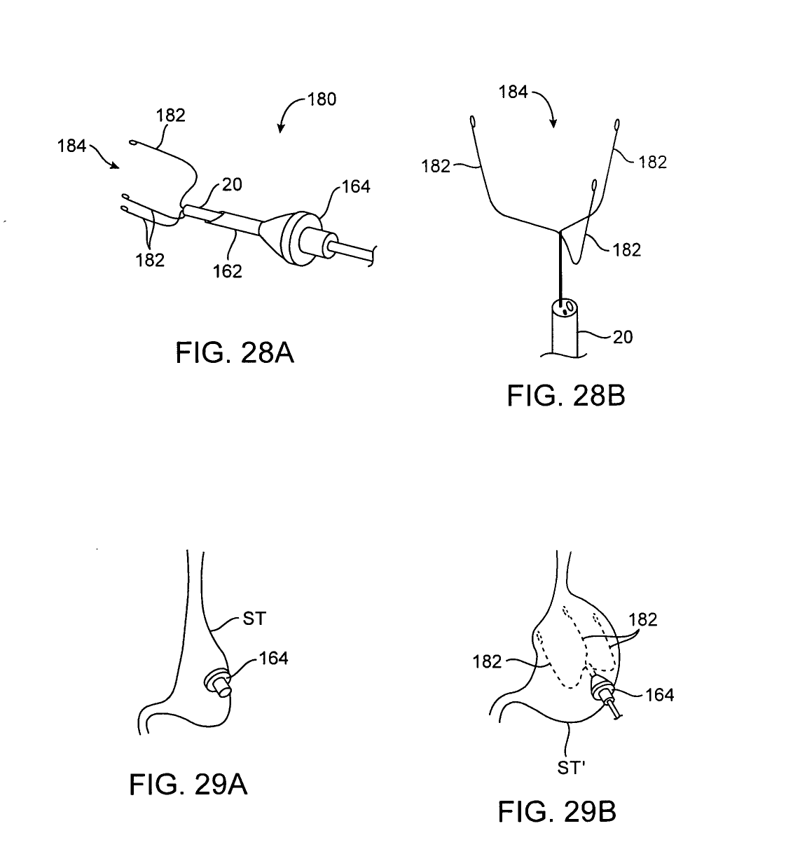

FIG. 28A shows another expandable apparatus 180 which may be delivered either transorally or transgastrically, depending upon the type of procedure to be utilized. This particular variation 180 may generally comprise multiple reconfigurable arms 182 which may expand from a low-profile delivery configuration to an expanded configuration where each of the arms are configured to extend or expand away from one another to create a working area or space 184 between the arms 182. As shown in FIG. 28B, which shows a perspective view of the expanded apparatus 180, the arms 182 may be configured to expand with the arms parallel or angled away or towards one another. Although three arms are shown in this variation, this is not intended to be limiting and any number of arms as practicable may be utilized. In this variation, however, the three arms 182 may be configured to deploy in a triangular pattern to maximize the open working area 184.

FIGS. 29A and 28B show how the apparatus 180 of FIG. 28A may be inserted into port 164, if utilized transgastrically, into the stomach ST. Once within stomach ST, arms 182 may be allowed to reconfigure to extend the stomach ST′ open and to thus allow the tissue region of interest to be exposed for further procedures.

Although various illustrative embodiments are described above, it will be evident to one skilled in the art that various changes and modifications are within the scope of the invention. It is intended in the appended claims to cover all such changes and modifications that fall within the true spirit and scope of the invention.

1. An apparatus for displacing tissue within a lumen, comprising:
a delivery device which defines at least one channel therethrough; and
a elongate member adapted to have a first configuration when positioned within the channel and to self-expand into a second configuration when deployed from the channel into the lumen,
wherein the elongate member is further adapted to contact and displace the tissue when in the second configuration.

2. The apparatus of claim 1 wherein the delivery device comprises an endoscope defining at least one working lumen therethrough.

3. The apparatus of claim 1 wherein the delivery device comprises an articulatable distal section.

4. The apparatus of claim 1 wherein the delivery device comprises an elongate member defining at least one lumen therethrough selected from the group consisting of tubes, sheaths, and catheters.

5. The apparatus of claim 1 wherein the elongate member comprises a ribbon or wire.

6. The apparatus of claim 1 wherein the elongate member is comprised of a shape memory alloy.

7. The apparatus of claim 6 wherein the shape memory alloy comprises Nitinol.

8. The apparatus of claim 1 wherein the second configuration comprises a helix.

9. The apparatus of claim 8 wherein the helix is conically shaped.

10. The apparatus of claim 1 wherein the second configuration comprises multiple curved sections patterned in an alternating manner around bend portions.

11. The apparatus of claim 1 wherein the second configuration comprises a spherical or ellipsoid shape with a plurality of expandable arms or struts joined proximally and distally at respective attachment points.

12. The apparatus of claim 1 wherein the second configuration comprises a mesh configured into a conical shape.

13. The apparatus of claim 1 wherein the elongate member is adapted to be withdrawn proximally within the channel.

14. The apparatus of claim 1 wherein the elongate member comprises an atraumatic distal end.

15. The apparatus of claim 14 wherein the distal end is blunted.

16. The apparatus of claim 14 wherein the distal end is looped.

17. The apparatus of claim 14 wherein the distal end is capped.

18. The apparatus of claim 1 wherein the elongate member comprises a distal end adapted to be anchored onto the tissue.

19. The apparatus of claim 18 wherein the distal end is adapted to adhere to the tissue via a fastener selected from the group consisting of helixes, graspers, hooks, barbs, and vacuum.

20. A method for displacing tissue within a lumen, comprising:
positioning a delivery device proximally of or within the lumen; and
advancing an elongate member through the delivery device such that the elongate member self-expands into an expanded configuration and displaces the tissue within the lumen.

21. The method of claim 20 wherein positioning the delivery device comprises advancing an endoscope defining at least one working lumen therethrough.

22. The method of claim 20 wherein positioning the delivery device comprises manipulating an articulatable section of the delivery device to position a distal end of the delivery device relative to the lumen.

23. The method of claim 20 wherein advancing the elongate member comprises advancing a ribbon or wire through the delivery device.

24. The method of claim 20 wherein the elongate member self-expands into a helix shape.

25. The method of claim 20 wherein the elongate member self-expands into multiple curved sections patterned in an alternating manner around bend portions.

26. The method of claim 20 wherein the elongate member self-expands into a spherical or ellipsoid shape having a plurality of expandable arms or struts joined proximally and distally at respective attachment points.

27. The method of claim 20 further comprising proximally withdrawing the elongate member within the delivery device.

28. The method of claim 20 further comprising withdrawing the delivery device from the lumen while advancing the elongate member through the delivery device.

29. The method of claim 28 advancing the elongate member further comprises anchoring a distal end of the elongate member to the tissue.

30. The method of claim 20 wherein positioning the delivery device comprises advancing the delivery device transorally.

31. The method of claim 20 wherein positioning the delivery device comprises positioning the delivery device proximally of or within a stomach.
